(19) United States
(12) Patent Application Publication
(10) Pub. No.: US 2003/0224372 A1
(43) Pub. Date: Dec. 4, 2003

(54) Method for determining ethnic origin by means of STR profile

(76) Inventor: Denise Syndercombe-Court, London (GB)

Correspondence Address: ROSENTHAL & OSHA L.L.P., 1221 MCKINNEY AVENUE, SUITE 2800, HOUSTON, TX, 77010, US

(21) Appl. No.: 10/160,436

(22) Filed: May 31, 2002

(51) Int. Cl. C12Q 1/68

(52) U.S. Cl. 435/6

(57) ABSTRACT

A method is provided which enables the identification of the ethnic origin of a human subject by means of analysis of certain short tandem repeat (STR) markers from the Y-chromosome of the subject.

The present invention relates to the use of STR profiling to determine the ethnic origin of an individual.

The variation in short tandem repeat (STR) allele proportions between ethnic populations has been described previously (Kimpton et al PCR Methods Appl, 3 (13) 13-22 (1993); Bowcock et al Nature 368 455-457 (1994); Gill, P., & Evett, I., Genetica 96 69-87 (1995); Evett et al Int. J. Legal Med. 110 5-9 (1997); Kaska et at Electrophoresis 18 1620-1623 (1997); Wilson-Wilde et al Electrophoresis 18 1592-1597 (1997); Grasemann et al Hum. Hered. 49 139-141 (1999); Chakrabarty et al Electrophoresis 20 1682-1696 (2000); Wiegand et al Electrophoresis 21 889-895 (2000); Meyer et al Int. J. Legal Med. 107 314-322 (1995). These differences have suggested the basis for a system of ethnic profiling (Kimpton et at PCR Methods Appl. 3 (13)13-22 (1993).

Since 1995, in the UK, the Forensic Science Service has used six STR loci and a sex-determining locus to profile DNA samples for the National DNA database. The loci HUMVWFA31/A, HUMTH01, HUMFIBRA, D8S1179, D21S11, D18S51 and the X-Y homologous gene amelogenin have been used to compose the multiplex used in practice (Kimpton et al Electrophoresis 17 1283-1293 (1996). More recently the Forensic Science Service has supplemented its database with additional loci for greater discrimination: D3S1368, D19S433, D16S539 and D2S1338 (Cotton et al Forensic Science International 112 151-161 (2000). Information obtained from the analysis of samples of crime scenes has enabled the preparation of the National DNA database.

Scientists have made attempts in the past to predict racial origin from genetic markers found in blood and DNA. Some blood group markers, such as Fy^{o} and R^{o}, are prevalent in the black population, but can only be recognised amongst individuals who inherit the markers from both parents. Other markers, such as EAP(R), while being found more often amongst blacks, are not common enough to be of much use. Little is available amongst the various markers found in blood to help distinguish South Asians from others. Additionally, for forensic utility, any method should be able to make use of minute quantities of material. Most recently, six autosomal STRs have been used to infer ethnic origin (Lowe et at Forensic Sci. Int. 11 (1) 17-22 (2001)), correctly predicting 56%, 67% and 43% of the Caucasian, Afro-Caribbean and South Asians they tested. They hypothesised that it would be more difficult to distinguish Caucasians and South Asians than other groupings

Despite these achievements, there remains a need for an accurate means for determining the ethnic origin of an individual. Such methods would have tremendous impact in such areas as crime detection, paternity testing and for counselling and information for future adoptive parents.

It has now been found that such methods of ethnic profiling in humans can be surprisingly improved by the selection of STR markers from a non-autosomal chromosome, e.g. the Y-chromosome in the case of male individuals. This is unexpected since most STR's found to date on the Y-chromosome exhibit much lower levels of polymorphism when compared to autosomal STRs.

According to a first aspect of the invention, there is provided a method of identifying the ethnic origin of a human subject, the method comprising assaying a biological sample from the subject for the presence of at least three short tandem repeat (STR) markers in the Y-chromosome DNA of the subject, wherein the at least three STR markers are DYS438, DYS385 and DYS390 and the ethnic origin is one of Caucasian, afro-caribbean (african-american), or south asian.

The method of identifying the ethnic origin of an individual according to a method of the present invention can utilise a data-mining method, whereby large amounts of data are subjected to an analytic process that searches for systematic relationships between particular features. Each derived pattern is tested against new data sets until a robust model is identified.

Caucasian is generally accepted as defining populations of European origin. Afro-caribbean is a term used to define populations of African origin but now widely settled in the caribbean and in other areas of the world such as North and South America, and Europe. The term African-american is therefore also now widely used with regard to this population. South asian is a term used to describe populations whose origin is the Indian sub-continent.

The biological sample may be any sample that contains DNA. Since DNA is found within the nucleus of cells then the sample may be any material containing nucleated cellular material. Such material is the basis of all body tissues and may also be found freely floating within blood, sweat, saliva, semen, and any other bodily fluid in varying amounts. Various methods, such as freezing and thawing, or exposing the tissues to enzymatic digestion, can be used to free the nucleus of its cellular surroundings, providing DNA in its native form.

The method of assaying for the presence of an STR can use any convenient DNA amplification method, for example the polymerase chain reaction (PCR) whereby the double strand of the DNA molecule is disrupted by a heating process. Polymerase enzymes and nucleic acid substrates are provided to encourage a new complementary strand to develop and bind with the single stranded molecule ‘chain’ as the reaction mix cools. Each time the process is repeated the amount of DNA is doubled. The doubling process, or amplification, will become limited when the enzymes and substrates are exhausted, Encouragement for developing particular regions of the DNA molecule is provided by introducing short sequences of DNA that are complementary to and adjacent to the area of interest on the molecule, such that these will readily bind to the single stranded molecule as it cools, providing an enabling start to the production of the second strand. Later detection of these areas of interest within the molecule is facilitated with some form of detectable label, such as a fluorescent marker, introduced into the manufactured primer sequence.

Y-chromosome markers can provide additional benefits over autosomal STRs, for example, assisting in complex relationship studies and providing additional and more sensitive information about individuals involved in an allegation of rape that can be used for intelligence purposes.

Other useful markers include DYS19, DYS389-I, DYS389-II, DYS391, DYS392, DYS393, DYS437, DYS439. In a preferred embodiment of the invention, the method comprises the use of all eleven STR markers in the model development. In such methods the analysis may conveniently utilise a three multiplex approach to generate results by means of DNA amplification, although primers could be readily redesigned to provide multiplex combinations other than those described here. A pentaplex combination may be used to amplify five loci (for example DYS19, DYS389-I/II, DYS390, DYS393), and two triplex combinations to amplify the remainder (for example triplex no.1 can comprise DYS391, DYS437 and DYS439, where triplex no.2 can comprise DYS385, DYS392 and DYS438).)

The primer sequences for the STRs referred to above are:

| | |
|---|---|
| DYS385 | 5′ AGCATGGGTGACAGAGCTA 3′ |
| | 5′ GGGATGCTAGGTAAAGCTG 3′ |
| DYS392 | 5′ TCATTAATCTAGCTTTTAAAAACAA 3′ |
| | 5′ AGACCCATTTGATGCAATGT 3′ |
| DYS391 | 5′ CTATTCATTCAATCATACACCCA 3′ |
| | 5′ CTGGGAATAAAATCTCCCTGGTTGCAAG 3′ |
| DYS437 | 5′ GACTATGGGCGTGAGTGCAT 3′ |
| | 5′ AGACCCTGTCATTCACAGATGA 3′ |
| DYS438 | 5′ TGGGGAATAGTTGAACGGTAA 3′ |
| | 5′ GTGGCAGACGCCTATAATCC 3′ |
| DYS439 | 5′ TCCTGAATGGTACTTCCTAGGTTT 3′ |
| | 5′ GCCTGGCTTGGAATTCTTTT 3′ |
| DYS19 | 5′ CTACTGAGTTTCTGTTATAGT 3′ |
| | 5′ ATGGCATGTAGTGAGGACA 3′ |
| DYS389 I and II: | 5′ CCAACTCTCATCTGTATTATCTAT 3′ |
| | 5′ TCTTATCTCCACCCACCAGA 3′ |
| DYS390 | 5′ TATATTTTACACATTTTTGGGCC 3′ |
| | 5′ TGACAGTAAAATGAACACATTGC 3′ |
| DYS393 | 5′ GTGGTCTTCTACTTGTGTCAATAC 3′ |
| | 5′ AACTCAAGTCCAAAAAATGCGG 3′ |

Methods of the present invention may be used to assay a mixed population of human individuals to determine the ethnic origin of the subjects in the mixed population. The methods may also be used to identify an individual subject's ethnic origin by comparison to a local reference population or with respect to a control population.

Methods of assaying for STR's in the DNA of an individual include the polymerase chain reaction as described above. The marker DYS390 can accurately distinguish between a black (african-american) population and a mixed white/south asian population. The marker DYS438 can be used to father distinguish between the white and south asian populations. The marker DYS385 can be used to further refine the distinction between the white and south asian populations (and can also define a Japanese population from within these groups). The marker DYS385 will, in fact, provide alone a useful classification of individuals within a population into these ethnic groups. The additional markers help in refining the model to make it better.

Since the methods of the present invention utilise STR markers on the Y-chromosome, the subjects will in most cases be apparently male.

The STR markers used in accordance with this aspect of the invention provide a means for identifying the ethnic origin of an individual in which the statistical model used provides reasonably accurate results with the advantage of a simple assay being used with only three markers required. Other markers can of course be used to refine the results of such an analysis as described in accordance with the first aspect of the invention.

According to a second aspect of the invention, there is provided the use of short tandem repeat (STR) DYS385 as an indicator of ethnic origin of a human subject. This aspect of the invention also extends to a method of identifying the ethnic origin of a human subject, the method comprising assaying a biological sample from the subject for the presence of the short tandem repeat (STR) DYS385 in the DNA of the Y-chromosome of the subject, wherein the ethnic origin is one of caucasian, afro-caribbean (african-american), japanese or south asian.

Methods and uses in accordance with any aspect of the invention can also include the use of further STR markers as required. In some embodiments, the use of the autosomal STR marker Gc can be useful.

Preferred features for the second and subsequent aspects of the invention are as for the first aspect mutatis mutandis.

The invention will now be further described with reference to the following Examples which are present for the purposes of illustration only and are not to be construed as being limiting on the invention.

In the Examples, reference is made to a drawing, in which

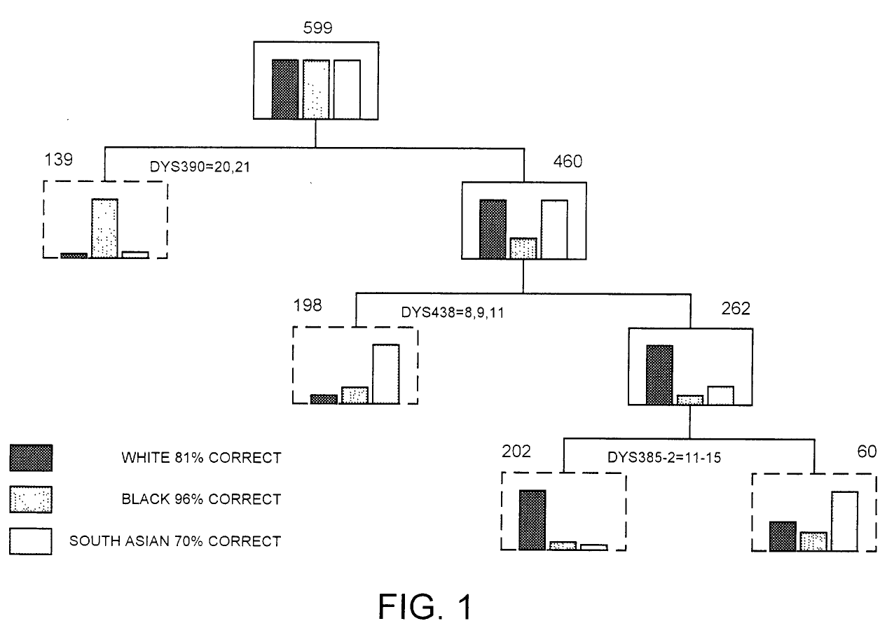

FIG. 1 shows the classification tree for ethnicity based on allelic markers. The total numbers in each classified group are shown above the box; within the box histograms illustrate the proportion identified in each group. The rule used for classification is shown between the boxes, with those individuals meeting the rule moving to the left. DYS385-2 refers to the large of the alleles in this biallelic marker (8,9,11 implies those alleles only, whereas 11-15 is meant to imply the range of alleles).

### MATERIALS AND METHODS

Methods for assaying for these particular STRs in the DNA of an individual with the use of the PCR includes the following protocols:

Triplex 1: DYS391, DYS437, DYS439 are amplified using steps 95° C. 10 minutes followed by a touchdown PCR with 8 cycles commencing with 94° C. 1 minute, 60° C. 1 minute, 72° C. 1 minute, each cycle reducing the annealing temperature by 0.5° C. This is followed by steps: 94° C. 1 minute, 56° C. 1 minute, 72° C. 1 minute for 22 cycles and a final step of 72° C. for 60 minutes. Primer concentrations are DYS391 0.25 μM, DYS437 0.4 μM and DYS439 0.25 μM, amplifying 1 ng of DNA.

Triplex 2: DYS385, DYS392, DYS438 are amplified using the same conditions as triplex 1 apart from the number of cycles for the final steps which are changed from 22 to 30. Primer concentrations are DYS385 0.2 μM, DYS392 0.5 μM and DYS438 0.3 μM, amplifying 2 ng of DNA.

Pentaplex: DYS 19, 389 I/II, 390 and 393 are amplified using steps 95° C. 10 minutes followed by 94° C. 1 minute, 55° C. 30 seconds, 72° C. 2 minutes for 28 cycles and a final step of 72° C. for 60 minutes.

Primer concentrations are DYS19 0.35 μM, DYS389 I/II 0.1 μM, DYS390 0.1 μM, and DYS393 0.15 μM, amplifying 5 ng of DNA.

An Applied Biosystems 310 automated sequencer in combination with Genescan™ 3.1 analysis software is used to detect and size the amplified fragments in comparison with sequenced allelic ladders, according to the International Society for Forensic Genetics (ISFG) guidelines for STR analysis (Gill et al International Journal of Legal Medicine 114 305-309 (2001)).

### EXAMPLE 1

Because of generally low levels of polymorphism, leading to poor individual discrimination, and the inherent linkage between the markers, profiles must be analysed as haplotypes, rather than as independent loci. We added three new markers (Ayub et at Nucleic Acids Research 28 e8 (2000)) to the standard eight (DYS 19, 385, 389-I, 389-II, 390, 391, 392, 393) to improve discrimination.

Six hundred male individuals were typed from the three ethnic groups most prevalent in the UK: Caucasians, and Afro-Caribbeans and South Asians.

Donors comprises mainly of individuals sampled for paternity analysis from mainland Britain supplemented by historic and ongoing collections of unrelated individuals. All donors provided consent and volunteer donors were made anonymous on collection for further protection. DNA was obtained from blood samples or mouth swabs and extracted using a standard Chelex method.

Three multiplexes (pentaplex, triplex I and triplex 2) were used to generate dye labelled products from the eleven loci.

An existing and widely used pentaplex combination amplified the loci: DYS 19, 389-I/II, 390 and 393 (Gusmao et al Forensic Science International 106 163-172 (1998)). Triplex 1 comprised DYS391, 437, and 439, which were amplified under the following conditions: 95° C. 15 minute, then 94° C. 1 minute, 60° C. 1 minute, 72° C. 1 minute using TouchDown PCR with eight cycles, each reducing the annealing temperature by 0.5° C., followed by 22 cycles of 94° C. 1 minute, 56° C. 1 minute, 72° C. 1 minute ending with 72° C. 5 min. Primer concentrations were: DYS391 0.25 μM, DYS437 0.4 μM, and DYS439 0.25 μM using 2 ng of DNA. Triplex 2 comprised DYS385, 392 and 438, which were amplified under the following conditions: 95° C. 15 minutes then 94° C. 1 minute, 72° C. 1 minute using TouchDown PCR with eight cycles, each reducing the annealing 60° C. 1 minute, temperature by 0.5° C., followed by 30 cycles of 94° C. 1 minute, 56° C. 1 minute, 72° C. 1 minute, ending with 72° C. 5 minutes. Primer concentrations were: DYS385 0.2 μM, DYS392 0.5 μM, and DYS438 0.3 μM using 2 ng of DNA. Allelic ladders were constructed for the three new loci, and all components were sequenced to confirm repeat number and absence of sequence anomalies.

Results

Locus diversity varied from a low of 0.28 (DYS392 in Afro-Caribbeans) to a high of 0.95 (DYS 385 in Afro-Caribbeans)

Haplotype diversities for the loci were 0.995 or more when the original eight loci were used, increasing to 0.999 and over with the additional loci.

Adding additional markers increased the proportion of distinct haplotypes observed by 11% to 92% in the Caucasian population, and by 5% each to 96% and 93% in the Afro-Caribbean and South Asian populations, respectively.

There were 29 shared haplotypes. Family names were available for 20 of these and shared only between one pair. Five haplotypes were shared between individuals from the separate Caucasian and Afro-Caribbean populations and one haplotype between individuals from the Caucasian and South Asian populations. There was no sharing observed between the Afro-Caribbean and South Asian groups.

Intermediate alleles were seen in 3/600 individuals: DYS385 (11-13.2), (14.2-18) and (14-16.2).

Duplications were seen in 2/600 individuals: DYS389-I and -II (13, 14 and 29, 30) seen in one individual, and DYS385 (11-14, 11-15). All anomalies were confirmed by repeat analysis and subsequent sequencing.

Discussion

Like autosomal STRs, intermediate alleles are sometimes observed and in this study were most often seen in the DYS385 paired allele locus. Duplications are seen at a similar frequency and, because many of the loci are closely linked, if observed, may be seen at more than one locus within an individual. Haplotype diversity is very high and across race group sharing is seen more often between Caucasians and Afro-Caribbeans than between Caucasians and South Asians, reflecting the social structure within the British population.

### EXAMPLE 2

Six hundred male individuals who described themselves and their parents as being “white”, “black”, or “from the Indian sub-continent (South Asian)”, 200 in each group, were typed for 11 Y-chromosome STRs (DYS 19, 385, 389-I/II, 390, 391, 392, 393, 437, 438 and 439). A further 159 individuals (around 50 from each group) were used for validation purposes. A data-mining approach based on the development of classification trees was used with selection of a tree based on lowest possible misclassification and simplicity of the tree (Breiman et al “Classification and Regression Trees”, Wadsworth & Brooks/Cole Advanced Books & Software, Monterey, Calif. (1984)).

TABLE 1
Likelihood ratios for competing hypotheses

| | Likelihood that the individual would have | | |
|---|---|---|---|
| | described himself as predicted compared with | | |
| | other groupings | | |
| Model prediction | White | Black | South Asian |
| White | | 10× | 4× |
| Black | 56× | | 34× |
| South Asian | 23× | 20× | |

Results

The selected classification model is illustrated in FIG. 1 and makes use of binary classifications to correctly classify 81% of white individuals, 96% of blacks, but only 70% of South Asians. In the model particular use is made of the common DYS390 (21) allele amongst black individuals. Three alleles in the DYS438 locus helped to identify some South Asians and more were identified with the DYS385 locus where South Asians are more represented amongst the larger alleles within the larger of the pair in this complex STR. Addition of Gc types to a subgroup of white and black individuals increased their correct classification of whites and blacks to 85% and 98%, respectively.

Table 1 illustrates the utility of the classification by presenting the competing likelihood ratios based on the best predictive model, For example, if the model predicts that the DNA is from someone who is“black” then the donor of that material is 56 times more likely to describe himself as “black” than “white”, and 34 times more likely to describe himself as “black” than “(south) asian”. In contrast, if the model predicts that the DNA is from someone who is “white”, then the donor of that material is only 10 times more likely to describe himself as being ‘white’ than ‘black’ and only 4 times more likely to describe himself as being ‘white’ than ‘(south) asian’.

Discussion

Use of a small constellation of Y-chromosome STR markers has produced a useful predictive ability for broad ethnic classification, particularly where the prediction is not “white”. Lowe et al predicted from Fst values that it would be more difficult to distinguish Caucasian from Asian, than Afro-Caribbean from Asian. Whilst this is true, this model has shown that prediction of someone as “white” has the least utility. The model has the lowest sensitivity (70%) for correctly identifying South Asians, compared with 96% for blacks and we are currently researching further markers to improve these and the former in particular. For example, incorporation of knowledge of the autosomal Gc type, will increase the correct classification of ‘white’ and ‘black’ individuals to 85% and 98% respectively. The predictive model has some important utility for intelligence purposes in particular, and has already proven useful in a social context. It should nevertheless be employed with caution. The model presented here has been validated with a UK based population and should be further validated with other populations where other markers may be more discriminating.

1. A method of identifying the ethnic origin of a human subject, the method comprising assaying a biological sample from the subject for the presence of at least three short tandem repeat (STR) markers in the DNA of the Y-chromosome of the subject, wherein the at least three STR markers are DYS438, DYS385 and DYS390 and the ethnic origin is one of caucasian, afro-caribbean, or south asian.

2. A method as claimed in claim 1, in which one or more STR markers selected from the group consisting of DYS437, DYS439, DYS19, DYS389-I, DYS389-II, DYS391, DYS392 and DYS393 are used in the identification of ethnic origin.

3. A method as claimed in claim 1 or claim 2, in which the autosomal STR marker Gc is used in the identification of ethnic origin.

4. The use of a short tandem repeat (STR) marker selected from the group consisting of DYS437, DYS438, DYS439, DYS385, DYS19, DYS389-I, DYS389-II DYS390, DYS391, DYS392 and DYS393 as a marker of ethnic origin of a human subject from a caucasian, afro caribbean, or south asian population.

5. The use the short tandem repeat (STR) DYS385 as an indicator of ethnic origin of a human subject from a caucasian, afro-caribbean (african-american), japanese or south asian population

6. A method of identifying the ethnic origin of a human subject, the method comprising assaying a biological sample from the subject for the presence of the short tandem repeat (STR) DYS385 in the DNA of the Y-chromosome of the subject, wherein the ethnic origin is one of Caucasian, afro-caribbean (african-american), japanese or south asian.

7. A method as claimed in the claim 6, in which the autosomal STR marker Gc is used in the identification of ethnic origin.
